(19) United States
(12) Patent Application Publication
(10) Pub. No.: US 2026/0297222 A1
(43) Pub. Date: Oct. 1, 2026

(54) POLYETHYLENE PRODUCTION FROM USED TIRES

(71) Applicant: Bridgestone Americas Tire Operations, LLC, Nashville, TN (US)

(72) Inventors: Terrence E. HOGAN, Uniontown, OH (US); Mark W. SMALE, Hudson, OH (US); William S. NIAURA, Hudson, OH (US)

(21) Appl. No.: 19/490,985

(22) PCT Filed: Jun. 7, 2024
(86) PCT No.: PCT/US2024/033021

§ 371 Date: Dec. 8, 2025

Related U.S. Application Data
(60) Provisional application No. 63/506,751, filed on Jun. 7, 2023.

(51) Int. Cl. C08F 10/02; B60C 1/00; C07C 1/24; C07C 29/151; C08K 3/04; C10J 3/00

(52) U.S. Cl. CPC C08F 10/02; B60C 1/00; C07C 1/24; C07C 29/151; C08K 3/04; C10J 3/00; C10J 2300/0916; C10J 2300/0946

(57) ABSTRACT

The present invention relates to a pyrolysis apparatus for disposing a waste tire. More specifically, the present invention relates to a two-stage pyrolysis apparatus for disposing a waste tire, a method for disposing a waste tire by using the two-stage pyrolysis apparatus for disposing a waste tire, and a system therefor. According to the present invention, the two-stage pyrolysis apparatus for disposing a waste tire pyrolyzes a waste tire over two stages by using an auger reactor before using a fluidized-bed reactor which is used for disposing the existing waste tire in order to extract oil of which sulfur content is significantly reduced.

### FIELD OF THE INVENTION

Embodiments of the present invention are directed toward a process for converting used tires to polyethylene, particularly polyethylene waxes, that are useful in rubber formulations including those used in the manufacture of tire components.

### BACKGROUND OF THE INVENTION

Used tires include a significant amount of carbonaceous material. Recycling the carbon into other carbon-based materials presents numerous challenges due, at least in part, to the fact that a majority of the carbon is tied up in a vulcanized network. As a result, technologically efficient modes of recycling used tire is fairly limited and includes, for example, mechanically grinding the vulcanized rubber product and using the ground rubber for various uses such as filler within composites. With an eye toward carbon efficiency, other modes of recycling carbon are desired.

### SUMMARY OF THE INVENTION

One or more embodiments of the present invention provide a process comprising (a) providing a feedstock that includes carbonaceous materials; (b) gasifying the feedstock to produce a gaseous stream including carbon monoxide, hydrogen, and carbon dioxide; (c) converting at least a portion of the carbon monoxide, hydrogen, and carbon dioxide to ethanol; (d) converting at least a portion of the ethanol to ethylene; and (e) converting at least a portion of the ethylene to polyethylene oligomers.

Other embodiments of the present invention provide a tire component comprising a cured rubber matrix with filler and polyethylene wax dispersed therein, where the polyethylene wax is obtained by polymerizing ethylene synthesized from constituents of a gaseous stream that is obtained from the gasification of a feedstock including carbonaceous materials.

Still other embodiments of the present invention provide a tire including a tire component as provided above, where the tire includes greater than 40 wt %, or 50 wt %, or 60 wt %, or 70 wt %, or 80 wt %, or 90 wt %, or 99 wt % sustainable material.

### DETAILED DESCRIPTION OF ILLUSTRATIVE EMBODIMENTS

Embodiments of the invention are based, at least in part, on the discovery of a process for consuming used tires in the production of polyethylene, particularly polyethylene waxes. These polyethylene waxes can advantageously be included within rubber formulations including those that are used to manufacture tire components. Embodiments of the invention therefore provide a methodology for converting used tires back to useful tires or tire components. According to one or more embodiments, used tires are thermally decomposed to form a gaseous stream. One or more components of this gaseous stream are then converted to polyethylene waxes. In one or more embodiments, one or more components of the gaseous stream are converted to ethanol, and the ethanol is then converted to ethylene. The ethylene is then oligomerized to form the polyethylene wax.

### Process Overview—Polyethylene Wax Synthesis

As suggested above, the process of the present invention provides a feedstock, which may include tire feedstock, and the feedstock is converted, via thermal decomposition, to a gaseous stream that includes hydrogen, carbon monoxide, and optionally carbon dioxide. This gaseous stream, which may be referred to as synthesis gas or syngas, is then converted to ethanol by using, for example, biological fermentation processes. The ethanol is then converted to ethylene by using known processes. The ethylene is then oligomerized, again using known processes, to polyethylene wax.

### Characteristics of Feedstock

In one or more embodiments, the feedstock that is thermally decomposed to form the gaseous stream may include tire feedstock from used tires, which may also be referred to as used tire feedstock or simply tire feedstock. As the skilled person appreciates, tire feedstock may include vulcanized polymer, carbon black filler, silica, resins, oils, fibrous yarn, and metal. The vulcanized polymer may include the sulfur-crosslinked residue of natural rubber and/or one or more synthetic elastomers including diene polymers and copolymers. In one or more embodiments, the used tire feedstock may include shredded or otherwise ground tires with one or more constituents of the used tire removed. For example, the tire feedstock may be treated to remove metal by methods known in the art (e.g. magnetic separation). Alternatively, or in combination therewith, the used tire feedstock may be optionally treated to remove fibrous reinforcement such as fiber yarn or cord, which the skilled person understands is often found in conjunction with the vulcanized rubber within many tire components. Alternatively, or in combination with the foregoing, the used tire feedstock may be optionally treated to remove inorganic materials such as silica filler, which the skilled person appreciates is often found in used tire components. In any event, the tire feedstock can be processed into tire shreds, tire chips, or ground or crumb rubber and fed to the thermal decomposition unit.

In one or more embodiments, the tire feedstock is characterized by relatively low amounts of metal, which low amounts may result from pre-treatment of the tire feedstock to remove at least a portion of the metal that is typically present in used tires. In one or more embodiments, following pre-treatment of the used tires to remove metal, the tire feedstock may include less than 25 wt %, in other embodiments less than 15 wt %, and in other embodiments less than 1 wt % metal based on the entire weight of the feedstock fed to thermal decomposition in accordance with the present invention.

In one or more embodiments, the tire feedstock is characterized by relatively low amounts of fibrous yarn or cord, which low amounts may result from pre-treatment of the tire feedstock to remove at least a portion of the fibrous yarn or cord that is typically present in used tires. In one or more embodiments, following pre-treatment, the tire feedstock includes less than 5 wt %, in other embodiments less than 4 wt %, in other embodiments less than 3 wt %, in other embodiments less than 2 wt %, and in other embodiments less than 1 wt % fibrous yarn or cord based on the entire weight of the feedstock fed to thermal decomposition in accordance with the present invention.

In one or more embodiments, the tire feedstock is characterized by relatively low amounts of inorganic filler (e.g. silica), which low amounts may result from pre-treatment of the tire feedstock to remove at least a portion of the inorganic filler that is typically present in used tires. In one or more embodiments, the tire feedstock includes less than 30 wt %, in other embodiments less than 20 wt %, in other embodiments less than 10 wt %, and in other embodiments less than 5 wt % inorganic filler based on the entire weight of the feedstock fed to thermal decomposition in accordance with the present invention.

In one or more embodiments, the used tire feedstock includes tire remains from passenger tires. In other embodiments, the used tire feedstock includes tire remains from non-passenger tires such as, but not limited to, truck and bus tires, off-road vehicle tires, agricultural tires, and race tires.

In one or more embodiments, the used tires are mechanically treated (e.g. ground or shredded) to form a ground or shredded material (i.e. the feedstock is ground or shredded). This ground or shredded material (i.e. the tire feedstock), which may also be referred to as crumb, may be characterized by an advantageous compacted density. For example, the feedstock may have a compacted density of greater than 640 kg/m³, in other embodiments greater than 720 kg/m³, and in other embodiments greater than 770 kg/m³, where density is determined by ASTM D 698-07.

In one or more embodiments, the feedstock provided to the thermal decomposition unit includes used tires and optionally complementary feedstock. In one or more embodiments, the complementary feedstock, which may also be referred to as co-feed, includes carbonaceous materials other than the tire feed stock. Carbonaceous material refers to any carbon material whether in solid, liquid, gas, or plasma state. Non-limiting examples of carbonaceous materials include carbonaceous liquid product, industrial liquid recycle, municipal solid waste (MSW or msw) including municipal solid waste with higher biomass content and/or with decreased recyclable material content, urban waste, agricultural material, forestry material, wood waste, construction material, vegetative material, industrial waste, fermentation waste, petrochemical coproducts, alcohol production coproducts, coal, plastics, waste plastic, coke oven tar, lignin, black liquor, polymers, waste polymers, polyethylene terephthalate (PETA), polystyrene (PS), sewage sludge, animal waste, crop residues, energy crops, forest processing residues, wood processing residues, livestock wastes, poultry wastes, food processing residues, ethanol coproducts, spent grain, spent microorganisms, municipal waste, construction waste, demolition waste, biomedical waste, hazardous waste, or their combinations. In one or more embodiments, the carbonaceous material includes biomass. In one or more embodiments, the biomass is bagasse including, but not limited to, the bagasse of sugar cane, sorghum, and guayule plant. In one or more embodiments, the feedstock includes a blend of used tires and municipal solid waste, wherein the municipal solid waste can include biomass. In other embodiments, the feedstock includes used tires and municipal solid waste that is substantially exclusive of biomass (i.e. substantially petroleum-based solid municipal waste). In yet other embodiments, the feedstock includes used tires and biomass. In still other embodiments, the feedstock includes used tires and municipal solid waste that has had most of the recyclable plastics removed (i.e. substantially exclusive of recyclable plastics). In sub-embodiments thereof, recyclable glass and metal is also substantially removed from the municipal solid waste component.

In a further embodiment, the guayule bagasse is produced as the result of a process to extract rubber and resin from the guayule plant, such as described in U.S. Publication No. 2022/0356273, which is incorporated herein by reference. Methods for the desolventization of guayule bagasse are described in U.S. Pat. No. 10,132,563, which is also incorporated herein by reference. In one or more embodiments, the guayule bagasse contains no more than 1 wt % organic solvent (based upon the total weight of the dried bagasse). In certain embodiments, the dried bagasse contains no more than 0.5 wt % organic solvent (based upon the total weight of the dried bagasse). In one or more embodiments, the dried bagasse may contain a quantity of water and higher boiling point terpenes. In certain embodiments, the total quantity of water and higher boiling point terpenes in the dried bagasse may be higher than the content of organic solvents.

In one or more embodiments, the co-feed (e.g. biomass or municipal waste) may be characterized by a compacted density of less than 600 kg/m³, in other embodiments less than 580 kg/m³, and in other embodiments less than 560 kg/m³, where density is determined by ASTM D 698-07.

The feedstock may be characterized by the amount of co-feed (e.g. biomass or municipal waste). In one or more embodiments, the feedstock includes from about 0 to about 95, in other embodiments from about 1 to about 75, and in other embodiments from about 2 to about 55 wt % co-feed with the balance including used tire. In one or more embodiments, the feedstock includes less than 95, in other embodiments less than 80, and in other embodiments less than 70 wt % co-feed. In these or other embodiments, the feedstock includes greater than 10, in other embodiments greater than 20, in other embodiments greater than 30, in other embodiments greater than 40, in other embodiments greater than 50, and in other embodiments greater than 70 wt % used tires, with the balance including complementary feedstock.

In alternative embodiments, the feedstock is substantially, and in certain embodiments exclusively, comprised of those carbonaceous materials identified above other than tire feedstock; i.e. the feedstock is substantially or exclusively comprised of the co-feed materials identified above. For example, in one or more embodiments, the feedstock includes greater than 80 wt %, in other embodiments greater than 90 wt %, and in other embodiments greater than 99 wt % municipal solid waste. Or, in other exemplary embodiments, the feedstock includes greater than 80 wt %, in other embodiments greater than 90 wt %, and in other embodiments greater than 99 wt % biomass.

### Thermal Decomposition of Feedstock

As indicated above, the feedstock (which may include tire feedstock and optionally co-feed) is thermally decomposed into gaseous streams including hydrogen, carbon monoxide, and optionally carbon dioxide by employing techniques that are generally known in the art. As the skilled person understands, these processes may include gasification processes, and it is also known that these processes can be tailored to control the chemical nature of the resulting gaseous stream. For example, the degree of combustion can be controlled by controlling the amount of oxygen present during thermal decomposition. In one or more embodiments, the step of thermal decomposition takes place in a substantially inert environment.

Processes that may be used for the thermal decomposition step may include pyrolysis or gasification reactions as disclosed in U.S. Publication Nos. 2021/0207037; 2019/0295734; 2019/0249089; 2018/0273415; 2017/0009162; 2017/0002271; 2016/0107913; 2016/0068773; 2016/0024404; 2014/0182205; 2014/0157667; and 2014/0100294, which are incorporated herein by reference.

In one or more embodiments, where the feedstock includes both tire feedstock and co-feed, the tire feedstock and the co-feed can be introduced to the same thermal decomposition unit simultaneously. For example, the tire feedstock and the co-feed can be pre-mixed at a desired ratio to form the feedstock that is fed to the thermal decomposition unit. Alternatively, separate streams of tire feedstock and co-feed can be separately and individually fed to the thermal decomposition unit at a desired rate. In yet other embodiment, the two feedstocks (i.e. the tire feedstock and the co-feed) can be sequentially treated within the same thermal decomposition unit. In still other embodiments, the two feedstocks (i.e. the tire feedstock and the co-feed) can be treated within separate thermal decomposition units operating in parallel, and then the gaseous streams produced by the respective units can be combined to attain the desired ratio of gaseous constituents.

### Characteristics of Gaseous Product Stream

As indicated above, the gaseous product stream produced by thermal decomposition of the feedstock includes carbon monoxide, hydrogen and optionally carbon dioxide. In one or more embodiments, the gaseous product stream includes from about 5 to about 50, or in other embodiments from about 7 to about 25, or in other embodiments from about 8 to about 15 volume percent carbon dioxide. In these or other embodiments, the gaseous product stream includes from about 10 to about 85, or in other embodiments from about 20 to about 65, or in other embodiments from about 25 to about 45 volume percent hydrogen. In these or other embodiments, the gaseous product stream includes from about 20 to about 85, or in other embodiments from about 30 to about 75, or in other embodiments from about 40 to about 60 volume percent carbon monoxide. In one or more embodiments, the gaseous product stream produced by thermal decomposition includes from about 40 to about 80 wt %, in other embodiments from about 45 to about 75 wt %, and in other embodiments from about 50 to about 70 wt % carbon (i.e. carbon within carbon-based compounds) based on the total weight of the gaseous product stream.

### Conditioning of Gaseous Stream

In one or more embodiments, the gaseous stream is conditioned (i.e. treated) prior to converting the gaseous stream to ethanol. In one or more embodiments, the gaseous product stream from thermal decomposition may be pressurized. In one or more embodiments, pressurization of the gaseous stream achieves sufficient pressure to overcome counter forces within the bioreactor. As the skilled person understands, this will permit flow of the gas through the bioreactor and allow inert gases (e.g. nitrogen) within the gaseous stream to enter the head space of the reactor. In one or more embodiments, the gaseous stream is pressurized to a pressure of from about 5 to about 20 barr.

Also, the gaseous stream can be cooled. As the skilled person will appreciate, the gaseous stream can be cooled, for example, within a heat exchanger such as a water-cooled unit. In one or more embodiments, the gaseous stream is cooled to a temperature below that which would otherwise have a deleterious impact on the microorganism culture within the bioreactor. In one or more embodiments, the gaseous stream is cooled to a temperature of from about 25 to about 45° C. prior to delivery to the bioreactor.

Still further, the gaseous stream can be treated to remove undesirable constituents that may be entrained within the stream. For example, the gaseous stream can be treated within a scrubber prior to being introduced to the bioreactor. In one or more embodiments, this may include the use of a catalyst (e.g. iron oxide) to remove sulfur compounds such as hydrogen sulfide. The stream may also be treated to remove acids (e.g. treatment with calcium or sodium carbonate with particular interest in removing hydrogen cyanides). Exemplary systems for removing hydrogen sulfide from gaseous streams include those available from EcoVapor (Denver, Colorado). Other methods include the use of caustic such as those systems available from DMT under the tradename Sulfurex BF (Netherlands).

### Syngas to Ethanol

As indicated above, constituents of the gaseous stream are converted to ethanol that is contained within an ethanol-containing stream; i.e. a product stream that includes ethanol. In one or more embodiments, the gaseous stream is supplemented with hydrogen prior to converting the gaseous stream to an ethanol-containing stream.

In one or more embodiments, the gaseous stream is converted to ethanol via biosynthetic techniques. For example, it is known that syngas can be converted to ethanol by fermentation utilizing microorganisms, such as bacteria. The microorganisms may be acetogenic autotrophic microbes. Acetogenic microbes (i.e. bacteria) generally transform the CO, H₂, and CO₂ in syngas into acetyl-CoA. The acetyl-CoA is then converted to organic products, such as acetic acid and ethanol. In one or more embodiments of the present invention, it may be desirable to preferentially produce ethanol relative to acetic acid.

As another potential pathway, some microorganisms (e.g. acetogens) can reduce the acetic acid (e.g. an organic acid) into an alcohol (e.g. ethanol). This acetogenic mechanism, as well as the microorganism chosen, may be considered relative to preferentially producing ethanol. Microorganisms other than acetogens may be suitable with different pathways to reaching ethanol. One or more useful microorganisms may simultaneous uptake both CO and H₂ in the syngas. In these or other embodiments, certain microorganisms can reduce CO2 to CO in the presence of excess hydrogen. For example, useful microorganisms and techniques for their use are disclosed in “A Techno-Economic Assessment of Bioethanol Production from Switchgrass Through Biomass Gasification and Syngas Fermentation” Regis et al, Energy 274, 127318, (2023).

The skilled person also appreciates that various conditions of microorganism-driven biosynthesis may be adjusted relative to preferential production of ethanol. For example, the pH, temperature, and concentrations of nutrients within the bioreactor where in the biosynthesis takes place can be adjusted. The desirable pH, temperature, and concentrations of nutrients may depend on the particular microorganisms employed.

In other embodiments, the syngas is converted to ethanol via thermochemical techniques. The skilled person understands that several thermochemical techniques exist to convert syngas to ethanol. For example, two-step processes exist whereby syngas can be converted to methanol using a hydrogen to carbon monoxide ratio of 2:1. These reactions typically take place in the gas phase using copper-based catalysts. The resulting product stream, which is typically saturated with water, can be purified by using known distillation techniques. The methanol can then be catalytically converted to ethanol. One-step catalytic techniques are also known. An exemplary thermochemical process for converting syngas to ethanol is described in U.S. Pat. No. 9,115,046, which is incorporated herein by reference.

As indicated above, the resultant ethanol is contained with an ethanol-containing stream. According to embodiments of the invention, a crude ethanol-containing stream, which is obtained directly from the bioreactor in which the ethanol is synthesized, is delivered to downstream steps. In other embodiments, the ethanol-containing stream is purified to produce an ethanol-containing stream that is higher in ethanol content. In one or more embodiments, the ethanol-containing stream exiting the step wherein syngas is converted to ethanol (i.e. the bioreactor) and delivered to the downstream steps where ethanol is further converted (optionally after purification) includes greater than 80 wt %, in other embodiments greater than 90 wt %, and in other embodiments greater than 95 wt % ethanol based upon the entire weight of the ethanol-containing stream.

### Ethanol to Ethylene

As indicated above, ethanol within the ethanol-containing stream is converted to ethylene, which is then contained within an ethylene-containing stream. In one or more embodiments, ethanol is converted to ethylene using catalytic techniques, which are well known in the art. Examples of useful catalysts include acid catalysts, alumina and transition metal oxides, silicoaluminophosphates (SAPO), HZSM-5 zeolite catalyst, and heteropolyacid catalysts. Modifications of these catalysts, such as nanoscale versions, may also be employed.

As the skilled person appreciates, some catalytic techniques proceed by dehydrating ethanol to form ethylene. For example, acid catalyst first protonates the hydroxyl group of the ethanol forming a molecule of water as a leaving group. The conjugate base of the remaining catalyst then deprotonates the methyl group, and the hydrocarbon rearranges to ethylene. In other embodiments, ethanol is converted to ethylene via catalytic dehydration over an aluminum oxide catalyst. In one or more embodiments, conversion of ethanol to ethylene takes place at a temperature of from about 180° C. to about 500° C.

The skilled person appreciates that processes for the conversion of ethanol to ethylene are well known and commercially available. For example, commercial processes are marketed by Braskem S.A., Technip Energies, Axens S.A. and Scientific Design Company, Inc.

The resultant ethylene is contained with an ethylene-containing stream. According to embodiments of the invention, a crude ethylene-containing stream, which is obtained directly from the reactor in which the ethylene is synthesized, is delivered to downstream steps. In other embodiments, the ethylene-containing stream is purified to produce an ethylene-containing stream that is higher in ethylene content. In one or more embodiments, the ethylene-containing stream exiting the step wherein ethanol is converted to ethylene and delivered to the downstream steps where ethylene is further converted (optionally after purification) includes greater than 80 wt %, in other embodiments greater than 90 wt %, and in other embodiments greater than 95 wt % ethylene based upon the entire weight of the ethylene-containing stream.

### Ethylene to Polyethylene Wax

Ethylene within the ethylene-containing stream is converted to polyethylene, which is then contained within a polyethylene-containing stream. The skilled person understands that the conversion of ethylene to polyethylene is accomplished by ethylene oligomerization. Techniques for oligomerizing ethylene are known. For example, ethylene monomer can be oligomerized in the presence of an appropriate catalyst while the ethylene is at least partially dissolved within an appropriate solvent. These polymerization techniques, which may be referred to as solution polymerization, may employ non-protic organic solvents such as, but not limited to, aliphatic hydrocarbons including hexanes. Useful catalysts include coordination catalysts such as a-nickel diamine catalysts, which are typically used in conjunction with alkyl aluminum co-catalysts. In other embodiments, Ziegler-Natta catalysts can be employed. In other embodiments, the ethylene within the ethylene-containing stream can be oligomerized by using gas-phase polymerization techniques. As the skilled person appreciates, these techniques may include use of fixed-bed polymerization systems. Exemplary processes for ethylene oligomerization are disclosed in “Alpha-diimine nickel complex, metal catalyst, branched polyethylene wax, and preparation method and application thereof,” CN 110511251B, published on Jan. 26, 2021, which is incorporated herein by reference.

In order to maintain a desirable polymerization medium, these reactions are typically maintained under pressure (e.g. pressures as high as 10 atmospheres), and may be conducted at temperatures of from about 20 to about 50° C.

Following oligomerization of the ethylene, the polyethylene wax can be recovered by employing known techniques for isolating the polyethylene wax from the polymerization mixture. For example, the polymerization mixture (i.e. polymer, solvent, and unreacted ethylene) can be desolventized by using known techniques. For example, the polymerization mixture can be steam desolventized to separate the polyethylene wax from the volatile constituents such as ethylene and solvent. In other embodiments, the wax can be separated by using a thin film evaporator.

The polyethylene wax produced by the present invention may be characterized by its Brookfield viscosity @125° C. In one or more embodiments, the polyethylene wax may have a Brookfield viscosity (125° C.) of from about 100 to about 3000, in other embodiments from about 500 to about 2500, and in other embodiments from about 1000 to about 2000 cP.

### INDUSTRIAL APPLICABILITY

In one or more embodiments, the polyethylene wax produced by the methods of this invention can be used in the production of tire components. As a result, practice of the present invention provides a method by which waste material, in particular waste material from used tires, is converted back to useful tires. In other words, a tire recycling or tire circularity method is provided. The wax synthesized and used in the practice of this invention may be referred to as circular synthetic wax.

Practice of the present invention not only offers a method for recycling tires by employing used tires as a feedstock to produce wax that can be formulated back into tires, but the practice of the present invention also advantageously provides a method whereby a tire is produced that has a relatively high content of sustainable constituents, which include recycled materials, naturally-derived materials and/or materials synthesized from bio-synthesized feedstock or bio-based materials. Moreover, these tires or tire components include threshold amounts of circular synthetic wax while being characterized by high sustainable content. For example, the tires or tire components of the present invention can include greater than 40 wt %, in other embodiments greater than 50 wt %, and in other embodiments greater than 60 wt % sustainable materials. In these or other embodiments, the tire or tire components include from about 40 to about 90 wt %, in other embodiments from about 45 to about 85 wt %, and in other embodiments from about 50 to about 80 wt % sustainable material. In combination therewith, the wax component of the tires or tire components of the present invention include greater than 10 wt %, in other embodiments greater than 20 wt %, in other embodiments greater than 30 wt %, in other embodiments greater than 40 wt %, in other embodiments greater than 45 wt %, and in other embodiments greater than 50 wt % circular synthetic wax, which includes synthetic wax produced according to embodiments of the present invention.

As the skilled person understands, tire components are produced by preparing and vulcanizing rubber compositions, which are often referred to as vulcanizable rubber compositions or simply rubber compositions. Generally, the vulcanizable rubber compositions include a rubber component, filler, wax, and curative. Other ingredients that may be included in the vulcanizable rubber composition include extender oils, processing oils, resins, coupling agents and antidegradants.

### Elastomer

The rubber component of the vulcanizable rubber compositions includes a vulcanizable elastomer, which may also be referred to as an elastomer, a vulcanizable rubber, or simply as a rubber. The skilled person appreciates that the vulcanizable elastomer is capable of being cured, which may also be referred to as vulcanized, to form an elastomeric composition. In one or more embodiments of this invention, the rubber component includes one or more synthetic polymers. These synthetic polymers may include, for example and without limitation, synthetic polyisoprene, polybutadiene, polyisobutylene-co-isoprene, neoprene, poly(ethylene-co-propylene), poly(styrene-co-butadiene), poly(styrene-co-isoprene), poly(styrene-co-isoprene-co-butadiene), poly(isoprene-co-butadiene), poly(ethylene-co-propylene-co-diene), polysulfide rubber, acrylic rubber, urethane rubber, silicone rubber, epichlorohydrin rubber, and mixtures thereof. These elastomers can have a myriad of macromolecular structures including linear, branched, and star-shaped structures.

In these or other embodiments, the rubber component of the vulcanizable rubber compositions of this invention includes one or more circular synthetic rubbers, which is vulcanizable rubber produced from used tires. These circular synthetic rubbers may be used alone as the rubber component or in combination with synthetic polymers.

In these or other embodiments, the rubber component may include one or more natural rubbers. Natural rubber may be used alone as the rubber component or in combination with synthetic polymers and/or circular synthetic rubber. As the skilled person understands, natural rubber is synthesized by and obtained from plant life. For example, natural rubber can be obtained from Hevea rubber trees, guayule shrub, gopher plant, mariola, rabbitbrush, milkweeds, goldenrods, pale Indian plantain, rubber vine, Russian dandelions, mountain mint, American germander, and tall bellflower.

Generally, the rubber compositions of this invention include from about 30 to about 65 wt %, in other embodiments from about 35 to about 60 wt %, and in other embodiments from about 40 to about 55 wt % elastomer, based on the total weight of the tire component.

### Filler

As suggested above, the rubber compositions include fillers such as organic and inorganic fillers. Examples of organic fillers include carbon black and starch. Examples of inorganic fillers include silica, aluminum hydroxide, magnesium hydroxide, mica, talc (hydrated magnesium silicate), and clays (hydrated aluminum silicates). In certain embodiments, a mixture of different fillers may be advantageously employed.

The amount of total filler employed in the rubber compositions can be up to about 150 parts by weight per 100 parts by weight of rubber (phr), with about 30 to about 125 phr, or about 40 to about 110 phr being typical. In certain embodiments the total filler content is greater than about 100 phr. In other embodiments, the total filler content is from about 50 to about 100 phr, and in in further embodiments from about 55 to about 95 phr.

Conventional carbon black can be used, which is generally known in the art. In one or more embodiments, carbon blacks include furnace blacks, channel blacks, and lamp blacks. More specific examples of carbon blacks include super abrasion furnace blacks, intermediate super abrasion furnace blacks, high abrasion furnace blacks, fast extrusion furnace blacks, fine furnace blacks, semi-reinforcing furnace blacks, medium processing channel blacks, hard processing channel blacks, conducting channel blacks, and acetylene blacks.

In particular embodiments, the carbon blacks may have a surface area (EMSA) of at least 20 m²/g and in other embodiments at least 35 m²/g; surface area values can be determined by ASTM D-1765 using the cetyltrimethylammonium bromide (CTAB) technique. The carbon blacks may be in a pelletized form or an unpelletized flocculent form. The preferred form of carbon black may depend upon the type of mixing equipment used to mix the rubber compound.

In one or more embodiments, carbon black can be sourced from a recycled material. Such recycled material can include reclaimed or recycled vulcanized rubber, whereby the vulcanized rubber is typically reclaimed from manufactured articles such as a pneumatic tire, an industrial conveyor belt, a power transmission belt, and a rubber hose. The recycled carbon black may be obtained by a pyrolysis process or other methods known for obtaining recycled carbon black. In an aspect, a recycled carbon black can be formed from incomplete combustion of recycled rubber feedstock or rubber articles. In another aspect, the recycled carbon black can be formed from the incomplete combustion of feedstock including oil resulting from the tire pyrolysis process. The carbon blacks utilized in the preparation of the vulcanizable elastomeric compositions can be in pelletized form or an unpelletized flocculent mass.

The amount of carbon black employed in the rubber compositions can be up to about 75 parts by weight per 100 parts by weight of rubber (phr), with about 5 to about 60 phr, or about 10 to about 55 phr being typical.

The rubber composition can further include filler in the form of one or more recycled rubbers in a particulate form. Recycled particulate rubber is typically broken down and reclaimed (or recycled) by any of a plurality of processes, which can include physical breakdown, grinding, chemical breakdown, devulcanization, cryogenic grinding, a combination thereof, etc. The term recycled particulate rubber can relate to both vulcanized and devulcanized rubber, where devulcanized recycle or recycled rubber (reclaim rubber) relates to rubber which has been vulcanized, ground into particulates and may have further undergone substantial or partial devulcanization. In an example, the recycled particulate rubber used in the rubber composition is essentially free of recycled rubber resulting from devulcanization. In a situation where the vulcanized rubber contains wire or textile fiber reinforcement, such wire or fiber reinforcement can be removed by any suitable process such as magnetic separation, air aspiration and/or air flotation step. In certain embodiments, the “recycled particulate rubber” comprises cured, i.e., vulcanized (crosslinked) rubber that has been ground or pulverized into particulate matter having a mean average particle size as discussed below.

Certain silicas may be considered sustainable materials. Some commercially available silicas which may be used as sustainable materials for the current invention include Hi-Sil™ 215, Hi-Sil™ 233, and Hi-Sil™ 190 (PPG Industries, Inc. ; Pittsburgh, Pa.). Other suppliers of commercially available silica include Grace Davison (Baltimore, Md.), Degussa Corp. (Parsippany, N.J.), Rhodia Silica Systems (Cranbury, N.J.), and J.M. Huber Corp. (Edison, N.J.). Other sustainable silicas include those derived from rice husk ash.

In one or more embodiments, silicas may be characterized by their surface areas, which give a measure of their reinforcing character. The Brunauer, Emmet and Teller (“BET”) method (described in J. Am. Chem. Soc., 1939, vol. 60, 2 p. 309-319) is a recognized method for determining the surface area. The BET surface area of silica is generally less than 450 m²/g. Useful ranges of surface area include from about 32 to about 400 m²/g, about 100 to about 250 m²/g, and about 130 to about 240 m²/g, and about 170 to about 220 m²/g. In certain embodiments, the silica may have a BET surface area of 190 to about 280 m²/g. The pH's of the silicas are generally from about 5 to about 7 or slightly over 7, or in other embodiments from about 5.5 to about 6.8.

In one or more embodiments, where silica is employed as a filler (alone or in combination with other fillers), a coupling agent and/or a shielding agent may be added to the rubber compositions during mixing in order to enhance the interaction of silica with the elastomers. Useful coupling agents and shielding agents are disclosed in U.S. Pat. Nos. 3,842,111; 3,873,489; 3,978,103; 3,997,581; 4,002,594; 5,580,919; 5,583,245; 5,663,396; 5,674,932; 5,684,171; 5,684,172; 5,696,197; 6,608,145; 6,667,362; 6,579,949; 6,590,017; 6,525,118; 6,342,552; and 6,683,135; which are incorporated herein by reference.

The amount of silica employed in the rubber compositions can be from about 1 to about 150 phr or in other embodiments from about 5 to about 130 phr. The useful upper range is limited by the high viscosity imparted by silicas. In certain embodiments, the silica employed in the rubber composition is derived from rice husk ash only, and in other embodiments the rubber compositions do not include silica from non-rice husk ash derived processes. When silica is used together with carbon black, the amount of the silica or carbon black individually can be as low as about 1 phr. Generally, the amounts of coupling agents and shielding agents range from about 4 wt % to about 20 wt % based on the weight of silica used. In one or more embodiments, where carbon black and silica are employed in combination as a filler, the weight ratio or silica to total filler may be from about 5 wt % to about 99 wt % of the total filler, in other embodiments from about 10 wt % to about 90 wt % of the total filler, or in yet other embodiments from about 50 wt % to about 85 wt % of the total filler. In certain embodiments the silica and carbon black fillers employed in the rubber composition are selected from the group consisting of sustainable pyrolysis carbon black and/or rice husk ash derived silica.

CURATIVE A multitude of rubber curing agents (also called vulcanizing agents) may be employed, including sulfur or peroxide-based curing systems. Curing agents are described in Kirk-Othmer, ENCYCLOPEDIA OF CHEMICAL TECHNOLOGY, Vol. 20, pgs. 365-468, (3^{rd} Ed. 1982), particularly Vulcanization Agents and Auxiliary Materials, pgs. 390-402, and A. Y. Coran, Vulcanization, ENCYCLOPEDIA OF POLYMER SCIENCE AND ENGINEERING, (2^{nd} Ed. 1989), which are incorporated herein by reference. Vulcanizing agents may be used alone or in combination.

### Circular Synthetic Wax

As indicated above, the rubber compositions of this invention include the circular synthetic wax disclosed above. In addition thereto, the rubber composition may include one or more natural waxes. A natural wax, or one with no petroleum as its raw material, can include carnauba wax, candelilla wax (e.g., extracted from candelilla flowers), rice wax (e.g., separated from rice bran oil) and Japan wax (e.g., extracted from Japanese wax tree). In these or other embodiments, the circular synthetic wax, optionally together with a natural wax, may also be used in conjunction petroleum-based waxes.

Generally, the rubber compositions of this invention include from about 1 to about 20 parts by weight, or in other embodiments from about 2 to about 15 parts by weight total wax per 100 parts by weight rubber. The amount of circular synthetic wax, relative to the total weight of wax included, may be from about 1 wt % to about 100 wt %, or in other embodiment from about 20 wt % to about 80 wt % of the total wax. In certain embodiments, the rubber composition includes circular synthetic waxes only.

In one or more embodiments, the rubber compositions of this invention include threshold amounts of sustainable waxes, which for purposes of this invention include circular synthetic waxes and natural waxes. The amount of sustainable wax, relative to the total weight of wax included, may be from about 1 wt % to about 99 wt %, or in other embodiment from about 20 wt % to about 80 wt % of the total wax. In certain embodiments, the rubber composition includes sustainable waxes only.

### Oils

With regard to oils, sustainable oils, which include plant-based oils and bio-based oils, may be used. Plant-based oils may include plant-based triglycerides. Exemplary oils include, without limitation, palm oil, soybean oil (also referred to herein as soy oil), rapeseed oil, sunflower seed, peanut oil, cottonseed oil, oil produced from palm kernel, coconut oil, olive oil, corn oil, grape seed oil, hemp oil, linseed oil, rice oil, safflower oil, sesame oil, mustard oil, flax oil. Other examples include nut-derived oils such oils obtained from beech nuts, cashews, mongongo nuts, macadamia nuts, pine nuts, hazelnuts, chestnuts, acorns, almonds, pecans, pistachios, walnuts, or brazil nuts. As the skilled person will appreciate, these oils can be produced by any suitable process such as mechanical extraction (e.g., using an oil mill), chemical extraction (e.g., using a solvent, such as hexane or carbon dioxide), pressure extraction, distillation, leaching, maceration, purification, refining, hydrogenation, sparging, etc.

Bio-based oils, also referred to as bio-oils, can include oils produced by a recombinant cell. For example, bio-oils produced by recombinant cells can be produced using a select strain of algal cells that are fed with a supply of sugars (e.g., sucrose) and then allowed to ferment and produce a bio-oil with a selected profile; after sufficient growth or fermentation has taken place, the bio-oil is isolated from the cells and collected.

Generally, the rubber compositions of this invention can include from about 1 to about 70 parts by weight, or in other embodiments from about 5 to about 50 parts weight total oil per 100 parts by weight rubber. The amount of sustainable oil, relative to the total weight of oil included, may be from about 1 wt % to about 99 wt %, or in other embodiment from about 20 wt % to about 80 wt %.

### Other Ingredients

Other ingredients that are typically employed in rubber compounding may also be added to the rubber compositions. These include accelerators, accelerator activators, oils, plasticizer, waxes, scorch inhibiting agents, processing aids, zinc oxide, tackifying resins, reinforcing resins, fatty acids such as stearic acid, peptizers, and antidegradants such as antioxidants and antiozonants.

### Rubber Composition Processing

All ingredients of the rubber compositions can be mixed with standard mixing equipment such as Banbury or Brabender mixers, extruders, kneaders, and two-rolled mills. In one or more embodiments, the ingredients are mixed in two or more stages. In the first stage (often referred to as the masterbatch mixing stage), a so-called masterbatch, which typically includes the rubber component and filler, is prepared. To prevent premature vulcanization (also known as scorch), the masterbatch may exclude vulcanizing agents. The masterbatch may be mixed at a starting temperature of from about 25° C. to about 125° C. with a discharge temperature of about 135° C. to about 180° C. Once the masterbatch is prepared, the vulcanizing agents may be introduced and mixed into the masterbatch in a final mixing stage, which is typically conducted at relatively low temperatures so as to reduce the chances of premature vulcanization. Optionally, additional mixing stages, sometimes called remills, can be employed between the masterbatch mixing stage and the final mixing stage. One or more remill stages are often employed where the rubber composition includes silica as the filler. Various ingredients including the polymers of this invention can be added during these remills.

The mixing procedures and conditions particularly applicable to silica-filled tire formulations are described in U.S. Pat. Nos. 5,227,425; 5,719,207; and 5,717,022, as well as European Patent No. 890,606, all of which are incorporated herein by reference. In one embodiment, the initial masterbatch is prepared by including the polymer and silica in the substantial absence of coupling agents and shielding agents.

### Tire Fabrication

In order to fabricate tire components with the polymers produced by the invention, the skilled person appreciates that the polymers are mixed with various other ingredients (e.g. filler and curative) to produce a rubber composition (also referred to as vulcanizable composition), and the vulcanizable composition is then processed into tire components according to ordinary tire manufacturing techniques, which generally include standard rubber shaping and molding techniques. The tire components may include, but are not limited to, tire treads, sidewalls, subtreads, body ply skims, and bead filler. The various tire components are then assembled into a green tire (i.e. an uncured tired), placed within a mold, and then vulcanized. Typically, vulcanization is effected by heating the vulcanizable composition in a mold; e.g., it may be heated to about 140° C. to about 180° C. Cured or crosslinked rubber compositions may be referred to as vulcanizates, which generally contain three-dimensional polymeric networks that are thermoset. The other ingredients, such as fillers and processing aids, may be evenly dispersed throughout the crosslinked network. Pneumatic tires can be made as discussed in U.S. Pat. Nos. 5,866,171; 5,876,527; 5,931,211; and 5,971,046, which are incorporated herein by reference.

### Tire Reinforcements

In one or more embodiments, the tires can include fabric reinforcement made by using non-petroleum materials in place of synthetic fibers. For example, mechanical recycled fibers, chemical recycled fibers, or bio-based fibers can be used. Likewise, the tires can include metal reinforcement made from recycled steel and/or other circular or sustainable metals. These non-petroleum fabrics and recycled metals can be used exclusively within the tires or in combination with traditional fabric and/or metal reinforcement.

Various modifications and alterations that do not depart from the scope and spirit of this invention will become apparent to those skilled in the art. This invention is not to be duly limited to the illustrative embodiments set forth herein.

1. A process comprising:
(a) providing a feedstock that includes carbonaceous materials;
(b) gasifying the feedstock to produce a gaseous stream including carbon monoxide, hydrogen, and carbon dioxide;
(c) converting at least a portion of the carbon monoxide, hydrogen, and carbon dioxide to ethanol;
(d) converting at least a portion of the ethanol to ethylene; and
(e) converting at least a portion of the ethylene to polyethylene oligomers.

2. The process of claim 1, where said step of converting at least a portion of the carbon monoxide, hydrogen, and carbon dioxide to ethanol includes biosynthetically converting at least a portion of the carbon monoxide, hydrogen, and carbon dioxide to ethanol, or where said the step of converting at least a portion of the carbon monoxide, hydrogen, and carbon dioxide to ethanol includes thermochemically converting at least a portion of the carbon monoxide, hydrogen, and carbon dioxide to ethanol.

3. The process of claim 1, where said step of converting at least a portion of the ethanol to ethylene includes catalytically dehydrating the ethanol.

4. The process of claim 1, where said step of converting at least a portion of the ethylene to polyethylene oligomers includes oligomerizing the ethylene within a solution polymerization.

5. The process of claim 1, where the feedstock includes used tire feedstock.

6. The process of claim 1, where the feedstock includes biomass.

7. The process of claim 1, where the feedstock includes municipal solid waste.

8. The process of claim 1, where the feedstock includes a mixture of tire feedstock and a complementary feedstock.

9. The process of claim 1, further comprising the step of fabricating a tire component with the polyethylene wax.

10. A tire component comprising:
a cured rubber matrix with filler and polyethylene wax dispersed therein, where the polyethylene wax is obtained by polymerizing ethylene synthesized from constituents of a gaseous stream that is obtained from the gasification of a feedstock including carbonaceous materials.

11. The tire component of claim 10, where the carbonaceous material includes used tire feedstock.

12. The tire component of claim 10, where the carbonaceous material includes biomass.

13. The tire component of claim 10, where the carbonaceous material includes solid municipal waste.

14. The tire component of claim 10, where the carbonaceous material includes a mixture of used tire feedstock together with a complementary feedstock.

15. The tire component of claim 10, where said tire component includes one or more additional constituents dispersed within the cured rubber matrix and one or more of these additional constituents are sustainable materials, and where greater than 40 wt %, or 50 wt %, or 60 wt %, or 70 wt %, or 80 wt %, or 90 wt %, or 99 wt % of the tire component includes circular synthetic rubber and sustainable materials.

16. The tire component of claim 10, where the filler includes recycled carbon black.

17. The tire component of any claim 10, where the filler includes silica and carbon black, and wherein the silica is rice husk ash derived silica, and where the carbon black is a pyrolysis carbon black.

18. The tire component of claim 10, where the tire component includes a sustainable oil dispersed within said rubber matrix.

19. A tire including the tire component of claim 10, where the tire includes greater than 40 wt %, or 50 wt %, or 60 wt %, or 70 wt %, or 80 wt %, or 90 wt %, or 99 wt % sustainable material.

20. The tire of claim 19, where the tire includes recycled metal.
